SEQUENCE LISTING (203 sequences)

SEQ ID NO: 1 - DNA, 12 bp, synthetic construct
  misc_feature 1..12: note = synthetic
  source 1..12: mol_type = other DNA
gctgtacggatt

SEQ ID NO: 2 - DNA, 20 bp, synthetic construct
  misc_feature 1..20: note = synthetic
  source 1..20: mol_type = other DNA
ccaccgctgagcaataacta

SEQ ID NO: 3 - DNA, 20 bp, synthetic construct
  misc_feature 1..20: note = synthetic
  source 1..20: mol_type = other DNA
cgtagatgagtcaacggcct

SEQ ID NO: 4 - DNA, 12 bp, synthetic construct
  misc_feature 1..12: note = synthetic
  source 1..12: mol_type = other DNA
gtagtgacaggt

SEQ ID NO: 5 - DNA, 12 bp, synthetic construct
  misc_feature 1..12: note = synthetic
  source 1..12: mol_type = other DNA
tctgtgaagtcc

SEQ ID NO: 6 - DNA, 12 bp, synthetic construct
  misc_feature 1..12: note = synthetic
  source 1..12: mol_type = other DNA
atcagatcgcct

SEQ ID NO: 7 - DNA, 12 bp, synthetic construct
  misc_feature 1..12: note = synthetic
  source 1..12: mol_type = other DNA
aatgtggtctcg

SEQ ID NO: 8 - DNA, 12 bp, synthetic construct
  misc_feature 1..12: note = synthetic
  source 1..12: mol_type = other DNA
cctctccaaaca

SEQ ID NO: 9 - DNA, 12 bp, synthetic construct
  misc_feature 1..12: note = synthetic
  source 1..12: mol_type = other DNA
tactggacaagg

SEQ ID NO: 10 - DNA, 12 bp, synthetic construct
  misc_feature 1..12: note = synthetic
  source 1..12: mol_type = other DNA
tatcggagtcct

SEQ ID NO: 11 - DNA, 12 bp, synthetic construct
  misc_feature 1..12: note = synthetic
  source 1..12: mol_type = other DNA
ggtggagttact

SEQ ID NO: 12 - DNA, 12 bp, synthetic construct
  misc_feature 1..12: note = synthetic
  source 1..12: mol_type = other DNA
cggctactattg

SEQ ID NO: 13 - DNA, 12 bp, synthetic construct
  misc_feature 1..12: note = synthetic
  source 1..12: mol_type = other DNA
ccgagctatgta

SEQ ID NO: 14 - DNA, 12 bp, synthetic construct
  misc_feature 1..12: note = synthetic
  source 1..12: mol_type = other DNA
actacgtccaac

SEQ ID NO: 15 - DNA, 12 bp, synthetic construct
  misc_feature 1..12: note = synthetic
  source 1..12: mol_type = other DNA
ttcatccgaacg

SEQ ID NO: 16 - DNA, 12 bp, synthetic construct
  misc_feature 1..12: note = synthetic
  source 1..12: mol_type = other DNA
cgaaacgcttag

SEQ ID NO: 17 - DNA, 12 bp, synthetic construct
  misc_feature 1..12: note = synthetic
  source 1..12: mol_type = other DNA
gcctaagttcca

SEQ ID NO: 18 - DNA, 12 bp, synthetic construct
  misc_feature 1..12: note = synthetic
  source 1..12: mol_type = other DNA
caattcccacgt

SEQ ID NO: 19 - DNA, 12 bp, synthetic construct
  misc_feature 1..12: note = synthetic
  source 1..12: mol_type = other DNA
cggtgagacata

SEQ ID NO: 20 - DNA, 12 bp, synthetic construct
  misc_feature 1..12: note = synthetic
  source 1..12: mol_type = other DNA
ctctgaggtttg

SEQ ID NO: 21 - DNA, 12 bp, synthetic construct
  misc_feature 1..12: note = synthetic
  source 1..12: mol_type = other DNA
tactgtcaccca

SEQ ID NO: 22 - DNA, 12 bp, synthetic construct
  misc_feature 1..12: note = synthetic
  source 1..12: mol_type = other DNA
caggaggtacat

SEQ ID NO: 23 - DNA, 12 bp, synthetic construct
  misc_feature 1..12: note = synthetic
  source 1..12: mol_type = other DNA
cttcctacagca

SEQ ID NO: 24 - DNA, 12 bp, synthetic construct
  misc_feature 1..12: note = synthetic
  source 1..12: mol_type = other DNA
tagaaaccgagg

SEQ ID NO: 25 - DNA, 12 bp, synthetic construct
  misc_feature 1..12: note = synthetic
  source 1..12: mol_type = other DNA
gaaaagcgtacc

SEQ ID NO: 26 - DNA, 12 bp, synthetic construct
  misc_feature 1..12: note = synthetic
  source 1..12: mol_type = other DNA
cgctcataactc

SEQ ID NO: 27 - DNA, 12 bp, synthetic construct
  misc_feature 1..12: note = synthetic
  source 1..12: mol_type = other DNA
ggcatatacgac

SEQ ID NO: 28 - DNA, 12 bp, synthetic construct
  misc_feature 1..12: note = synthetic
  source 1..12: mol_type = other DNA
gtgctctatcac

SEQ ID NO: 29 - DNA, 12 bp, synthetic construct
  misc_feature 1..12: note = synthetic
  source 1..12: mol_type = other DNA
ggagcatttcac

SEQ ID NO: 30 - DNA, 12 bp, synthetic construct
  misc_feature 1..12: note = synthetic
  source 1..12: mol_type = other DNA
atgggtcttctg

SEQ ID NO: 31 - DNA, 12 bp, synthetic construct
  misc_feature 1..12: note = synthetic
  source 1..12: mol_type = other DNA
aagtccgtgaac

SEQ ID NO: 32 - DNA, 12 bp, synthetic construct
  misc_feature 1..12: note = synthetic
  source 1..12: mol_type = other DNA
tgacatagaggg

SEQ ID NO: 33 - DNA, 12 bp, synthetic construct
  misc_feature 1..12: note = synthetic
  source 1..12: mol_type = other DNA
cgtcaatcgtgt

SEQ ID NO: 34 - DNA, 12 bp, synthetic construct
  misc_feature 1..12: note = synthetic
  source 1..12: mol_type = other DNA
gttcgaagcaac

SEQ ID NO: 35 - DNA, 12 bp, synthetic construct
  misc_feature 1..12: note = synthetic
  source 1..12: mol_type = other DNA
acccgaattcac

SEQ ID NO: 36 - DNA, 12 bp, synthetic construct
  misc_feature 1..12: note = synthetic
  source 1..12: mol_type = other DNA
gaggacttcaca

SEQ ID NO: 37 - DNA, 12 bp, synthetic construct
  misc_feature 1..12: note = synthetic
  source 1..12: mol_type = other DNA
gattccaccgta

SEQ ID NO: 38 - DNA, 12 bp, synthetic construct
  misc_feature 1..12: note = synthetic
  source 1..12: mol_type = other DNA
gtattcgccatg

SEQ ID NO: 39 - DNA, 12 bp, synthetic construct
  misc_feature 1..12: note = synthetic
  source 1..12: mol_type = other DNA
gcttgttatccg

SEQ ID NO: 40 - DNA, 12 bp, synthetic construct
  misc_feature 1..12: note = synthetic
  source 1..12: mol_type = other DNA
cgtccaactatg

SEQ ID NO: 41 - DNA, 12 bp, synthetic construct
  misc_feature 1..12: note = synthetic
  source 1..12: mol_type = other DNA
ggtaacagtgac

SEQ ID NO: 42 - DNA, 12 bp, synthetic construct
  misc_feature 1..12: note = synthetic
  source 1..12: mol_type = other DNA
gcgcaaaagaag

SEQ ID NO: 43 - DNA, 12 bp, synthetic construct
  misc_feature 1..12: note = syntehtic
  source 1..12: mol_type = other DNA
tgtggttgatcg

SEQ ID NO: 44 - DNA, 12 bp, synthetic construct
  misc_feature 1..12: note = synthetic
  source 1..12: mol_type = other DNA
tgtgggattgtg

SEQ ID NO: 45 - DNA, 12 bp, synthetic construct
  misc_feature 1..12: note = synthetic
  source 1..12: mol_type = other DNA
tgcttcgggata

SEQ ID NO: 46 - DNA, 12 bp, synthetic construct
  misc_feature 1..12: note = synthetic
  source 1..12: mol_type = other DNA
gacagctcgtta

SEQ ID NO: 47 - DNA, 12 bp, synthetic construct
  misc_feature 1..12: note = synthetic
  source 1..12: mol_type = other DNA
taagaagcgctc

SEQ ID NO: 48 - DNA, 12 bp, synthetic construct
  misc_feature 1..12: note = synthetic
  source 1..12: mol_type = other DNA
catacacactcc

SEQ ID NO: 49 - DNA, 12 bp, synthetic construct
  misc_feature 1..12: note = syntehtic
  source 1..12: mol_type = other DNA
tgccgccaaaat

SEQ ID NO: 50 - DNA, 12 bp, synthetic construct
  misc_feature 1..12: note = synthetic
  source 1..12: mol_type = other DNA
cggaccttctaa

SEQ ID NO: 51 - DNA, 12 bp, synthetic construct
  misc_feature 1..12: note = synthetic
  source 1..12: mol_type = other DNA
tctcacgtcaac

SEQ ID NO: 52 - DNA, 12 bp, synthetic construct
  misc_feature 1..12: note = synthetic
  source 1..12: mol_type = other DNA
cgcaagagaaca

SEQ ID NO: 53 - DNA, 12 bp, synthetic construct
  misc_feature 1..12: note = synthetic
  source 1..12: mol_type = other DNA
ttagcttccctg

SEQ ID NO: 54 - DNA, 12 bp, synthetic construct
  misc_feature 1..12: note = synthetic
  source 1..12: mol_type = other DNA
gaagccaagcat

SEQ ID NO: 55 - DNA, 12 bp, synthetic construct
  misc_feature 1..12: note = synthetic
  source 1..12: mol_type = other DNA
ttcgtagcgtgt

SEQ ID NO: 56 - DNA, 12 bp, synthetic construct
  misc_feature 1..12: note = synthetic
  source 1..12: mol_type = other DNA
gtcgctgatcaa

SEQ ID NO: 57 - DNA, 12 bp, synthetic construct
  misc_feature 1..12: note = synthetic
  source 1..12: mol_type = other DNA
tcaactgatcgg

SEQ ID NO: 58 - DNA, 12 bp, synthetic construct
  misc_feature 1..12: note = synthetic
  source 1..12: mol_type = other DNA
ccagtttctacg

SEQ ID NO: 59 - DNA, 12 bp, synthetic construct
  misc_feature 1..12: note = synthetic
  source 1..12: mol_type = other DNA
acccattgcgat

SEQ ID NO: 60 - DNA, 12 bp, synthetic construct
  misc_feature 1..12: note = synthetic
  source 1..12: mol_type = other DNA
tcaccaccctat

SEQ ID NO: 61 - DNA, 12 bp, synthetic construct
  misc_feature 1..12: note = synthetic
  source 1..12: mol_type = other DNA
ggtcttcacttc

SEQ ID NO: 62 - DNA, 12 bp, synthetic construct
  misc_feature 1..12: note = synthetic
  source 1..12: mol_type = other DNA
gttagagatggg

SEQ ID NO: 63 - DNA, 12 bp, synthetic construct
  misc_feature 1..12: note = synthetic
  source 1..12: mol_type = other DNA
tcttgcacactc

SEQ ID NO: 64 - DNA, 12 bp, synthetic construct
  misc_feature 1..12: note = synthetic
  source 1..12: mol_type = other DNA
ttttctctgcgg

SEQ ID NO: 65 - DNA, 12 bp, synthetic construct
  misc_feature 1..12: note = synthetic
  source 1..12: mol_type = other DNA
tcagccgagtta

SEQ ID NO: 66 - DNA, 12 bp, synthetic construct
  misc_feature 1..12: note = synthetic
  source 1..12: mol_type = other DNA
ctcgtgatcaga

SEQ ID NO: 67 - DNA, 12 bp, synthetic construct
  misc_feature 1..12: note = synthetic
  source 1..12: mol_type = other DNA
cctttctcggaa

SEQ ID NO: 68 - DNA, 12 bp, synthetic construct
  misc_feature 1..12: note = synthetic
  source 1..12: mol_type = other DNA
acgctagagctt

SEQ ID NO: 69 - DNA, 12 bp, synthetic construct
  misc_feature 1..12: note = synthetic
  source 1..12: mol_type = other DNA
ttccccgtttag

SEQ ID NO: 70 - DNA, 12 bp, synthetic construct
  misc_feature 1..12: note = synthetic
  source 1..12: mol_type = other DNA
agaatcgcaacc

SEQ ID NO: 71 - DNA, 12 bp, synthetic construct
  misc_feature 1..12: note = synthetic
  source 1..12: mol_type = other DNA
ggaaggaactgt

SEQ ID NO: 72 - DNA, 12 bp, synthetic construct
  misc_feature 1..12: note = synthetic
  source 1..12: mol_type = other DNA
cttggcatcttc

SEQ ID NO: 73 - DNA, 12 bp, synthetic construct
  misc_feature 1..12: note = synthetic
  source 1..12: mol_type = other DNA
aggccgatttgt

SEQ ID NO: 74 - DNA, 12 bp, synthetic construct
  misc_feature 1..12: note = synthetic
  source 1..12: mol_type = other DNA
aacaaagggtcc

SEQ ID NO: 75 - DNA, 12 bp, synthetic construct
  misc_feature 1..12: note = synthetic
  source 1..12: mol_type = other DNA
caattggtagcc

SEQ ID NO: 76 - DNA, 12 bp, synthetic construct
  misc_feature 1..12: note = synthetic
  source 1..12: mol_type = other DNA
accatcgactca

SEQ ID NO: 77 - DNA, 12 bp, synthetic construct
  misc_feature 1..12: note = synthetic
  source 1..12: mol_type = other DNA
cgtgagatgaac

SEQ ID NO: 78 - DNA, 12 bp, synthetic construct
  misc_feature 1..12: note = synthetic
  source 1..12: mol_type = other DNA
ccatggtcttgt

SEQ ID NO: 79 - DNA, 13 bp, synthetic construct
  misc_feature 1..13: note = synthetic
  source 1..13: mol_type = other DNA
cagatatgagcgc

SEQ ID NO: 80 - DNA, 12 bp, synthetic construct
  misc_feature 1..12: note = synthetic
  source 1..12: mol_type = other DNA
gtgtgacagagt

SEQ ID NO: 81 - DNA, 12 bp, synthetic construct
  misc_feature 1..12: note = synthetic
  source 1..12: mol_type = other DNA
attgtgtgacgg

SEQ ID NO: 82 - DNA, 12 bp, synthetic construct
  misc_feature 1..12: note = synthetic
  source 1..12: mol_type = other DNA
cggtagtttgct

SEQ ID NO: 83 - DNA, 12 bp, synthetic construct
  misc_feature 1..12: note = synthetic
  source 1..12: mol_type = other DNA
ggacatgtccat

SEQ ID NO: 84 - DNA, 12 bp, synthetic construct
  misc_feature 1..12: note = synthetic
  source 1..12: mol_type = other DNA
ttgagggagaca

SEQ ID NO: 85 - DNA, 12 bp, synthetic construct
  misc_feature 1..12: note = synthetic
  source 1..12: mol_type = other DNA
cgacatcctcta

SEQ ID NO: 86 - DNA, 12 bp, synthetic construct
  misc_feature 1..12: note = synthetic
  source 1..12: mol_type = other DNA
tgagcgagttca

SEQ ID NO: 87 - DNA, 12 bp, synthetic construct
  misc_feature 1..12: note = synthetic
  source 1..12: mol_type = other DNA
gaccttcggatt

SEQ ID NO: 88 - DNA, 12 bp, synthetic construct
  misc_feature 1..12: note = synthetic
  source 1..12: mol_type = other DNA
tgtagatccgca

SEQ ID NO: 89 - DNA, 12 bp, synthetic construct
  misc_feature 1..12: note = synthetic
  source 1..12: mol_type = other DNA
tggcactctaga

SEQ ID NO: 90 - DNA, 12 bp, synthetic construct
  misc_feature 1..12: note = synthetic
  source 1..12: mol_type = other DNA
aacagtagtcgg

SEQ ID NO: 91 - DNA, 12 bp, synthetic construct
  misc_feature 1..12: note = synthetic
  source 1..12: mol_type = other DNA
tcatgcggaaag

SEQ ID NO: 92 - DNA, 12 bp, synthetic construct
  misc_feature 1..12: note = synthetic
  source 1..12: mol_type = other DNA
tcgaatcgtgtc

SEQ ID NO: 93 - DNA, 12 bp, synthetic construct
  misc_feature 1..12: note = synthetic
  source 1..12: mol_type = other DNA
ggtgtatagcca

SEQ ID NO: 94 - DNA, 12 bp, synthetic construct
  misc_feature 1..12: note = synthetic
  source 1..12: mol_type = other DNA
ttgcagtgcaag

SEQ ID NO: 95 - DNA, 12 bp, synthetic construct
  misc_feature 1..12: note = synthetic
  source 1..12: mol_type = other DNA
cgattgcagaag

SEQ ID NO: 96 - DNA, 12 bp, synthetic construct
  misc_feature 1..12: note = synthetic
  source 1..12: mol_type = other DNA
ccagacgttgtt

SEQ ID NO: 97 - DNA, 12 bp, synthetic construct
  misc_feature 1..12: note = synthetic
  source 1..12: mol_type = other DNA
tggtggccataa

SEQ ID NO: 98 - DNA, 12 bp, synthetic construct
  misc_feature 1..12: note = synthetic
  source 1..12: mol_type = other DNA
cagagtcaatgg

SEQ ID NO: 99 - DNA, 12 bp, synthetic construct
  misc_feature 1..12: note = synthetic
  source 1..12: mol_type = other DNA
cctatcattccc

SEQ ID NO: 100 - DNA, 12 bp, synthetic construct
  misc_feature 1..12: note = synthetic
  source 1..12: mol_type = other DNA
gaggtatgactc

SEQ ID NO: 101 - DNA, 12 bp, synthetic construct
  misc_feature 1..12: note = synthetic
  source 1..12: mol_type = other DNA
ctaggtcaagtc

SEQ ID NO: 102 - DNA, 12 bp, synthetic construct
  misc_feature 1..12: note = synthetic
  source 1..12: mol_type = other DNA
actcggctttca

SEQ ID NO: 103 - DNA, 12 bp, synthetic construct
  misc_feature 1..12: note = synthetic
  source 1..12: mol_type = other DNA
ttcacaagcgga

SEQ ID NO: 104 - DNA, 52 bp, synthetic construct
  misc_feature 1..52: note = synthetic
  source 1..52: mol_type = other DNA
ccaccgctgagcaataactagtagtgacaggtcgtagatgagtcaacggcct

SEQ ID NO: 105 - DNA, 52 bp, synthetic construct
  misc_feature 1..52: note = synthetic
  source 1..52: mol_type = other DNA
ccaccgctgagcaataactatctgtgaagtcccgtagatgagtcaacggcct

SEQ ID NO: 106 - DNA, 52 bp, synthetic construct
  misc_feature 1..52: note = synthetic
  source 1..52: mol_type = other DNA
ccaccgctgagcaataactaatcagatcgcctcgtagatgagtcaacggcct

SEQ ID NO: 107 - DNA, 52 bp, synthetic construct
  misc_feature 1..52: note = synthetic
  source 1..52: mol_type = other DNA
ccaccgctgagcaataactaaatgtggtctcgcgtagatgagtcaacggcct

SEQ ID NO: 108 - DNA, 52 bp, synthetic construct
  misc_feature 1..52: note = synthetic
  source 1..52: mol_type = other DNA
ccaccgctgagcaataactacctctccaaacacgtagatgagtcaacggcct

SEQ ID NO: 109 - DNA, 52 bp, synthetic construct
  misc_feature 1..52: note = synthetic
  source 1..52: mol_type = other DNA
ccaccgctgagcaataactatactggacaaggcgtagatgagtcaacggcct

SEQ ID NO: 110 - DNA, 52 bp, synthetic construct
  misc_feature 1..52: note = synthetic
  source 1..52: mol_type = other DNA
ccaccgctgagcaataactatatcggagtcctcgtagatgagtcaacggcct

SEQ ID NO: 111 - DNA, 52 bp, synthetic construct
  misc_feature 1..52: note = synthetic
  source 1..52: mol_type = other DNA
ccaccgctgagcaataactaggtggagttactcgtagatgagtcaacggcct

SEQ ID NO: 112 - DNA, 52 bp, synthetic construct
  misc_feature 1..52: note = synthetic
  source 1..52: mol_type = other DNA
ccaccgctgagcaataactacggctactattgcgtagatgagtcaacggcct

SEQ ID NO: 113 - DNA, 52 bp, synthetic construct
  misc_feature 1..52: note = synthetic
  source 1..52: mol_type = other DNA
ccaccgctgagcaataactaccgagctatgtacgtagatgagtcaacggcct

SEQ ID NO: 114 - DNA, 52 bp, synthetic construct
  misc_feature 1..52: note = synthetic
  source 1..52: mol_type = other DNA
ccaccgctgagcaataactaactacgtccaaccgtagatgagtcaacggcct

SEQ ID NO: 115 - DNA, 52 bp, synthetic construct
  misc_feature 1..52: note = synthetic
  source 1..52: mol_type = other DNA
ccaccgctgagcaataactattcatccgaacgcgtagatgagtcaacggcct

SEQ ID NO: 116 - DNA, 52 bp, synthetic construct
  misc_feature 1..52: note = synthetic
  source 1..52: mol_type = other DNA
ccaccgctgagcaataactacgaaacgcttagcgtagatgagtcaacggcct

SEQ ID NO: 117 - DNA, 52 bp, synthetic construct
  misc_feature 1..52: note = synthetic
  source 1..52: mol_type = other DNA
ccaccgctgagcaataactagcctaagttccacgtagatgagtcaacggcct

SEQ ID NO: 118 - DNA, 52 bp, synthetic construct
  misc_feature 1..52: note = synthetic
  source 1..52: mol_type = other DNA
ccaccgctgagcaataactacaattcccacgtcgtagatgagtcaacggcct

SEQ ID NO: 119 - DNA, 52 bp, synthetic construct
  misc_feature 1..52: note = synthetic
  source 1..52: mol_type = other DNA
ccaccgctgagcaataactacggtgagacatacgtagatgagtcaacggcct

SEQ ID NO: 120 - DNA, 52 bp, synthetic construct
  misc_feature 1..52: note = synthetic
  source 1..52: mol_type = other DNA
ccaccgctgagcaataactactctgaggtttgcgtagatgagtcaacggcct

SEQ ID NO: 121 - DNA, 52 bp, synthetic construct
  misc_feature 1..52: note = synthetic
  source 1..52: mol_type = other DNA
ccaccgctgagcaataactatactgtcacccacgtagatgagtcaacggcct

SEQ ID NO: 122 - DNA, 52 bp, synthetic construct
  misc_feature 1..52: note = synthetic
  source 1..52: mol_type = other DNA
ccaccgctgagcaataactacaggaggtacatcgtagatgagtcaacggcct

SEQ ID NO: 123 - DNA, 52 bp, synthetic construct
  misc_feature 1..52: note = synthetic
  source 1..52: mol_type = other DNA
ccaccgctgagcaataactacttcctacagcacgtagatgagtcaacggcct

SEQ ID NO: 124 - DNA, 52 bp, synthetic construct
  misc_feature 1..52: note = synthetic
  source 1..52: mol_type = other DNA
ccaccgctgagcaataactatagaaaccgaggcgtagatgagtcaacggcct

SEQ ID NO: 125 - DNA, 52 bp, synthetic construct
  misc_feature 1..52: note = synthetic
  source 1..52: mol_type = other DNA
ccaccgctgagcaataactagaaaagcgtacccgtagatgagtcaacggcct

SEQ ID NO: 126 - DNA, 52 bp, synthetic construct
  misc_feature 1..52: note = synthetic
  source 1..52: mol_type = other DNA
ccaccgctgagcaataactacgctcataactccgtagatgagtcaacggcct

SEQ ID NO: 127 - DNA, 52 bp, synthetic construct
  misc_feature 1..52: note = synthetic
  source 1..52: mol_type = other DNA
ccaccgctgagcaataactaggcatatacgaccgtagatgagtcaacggcct

SEQ ID NO: 128 - DNA, 52 bp, synthetic construct
  misc_feature 1..52: note = synthetic
  source 1..52: mol_type = other DNA
ccaccgctgagcaataactagtgctctatcaccgtagatgagtcaacggcct

SEQ ID NO: 129 - DNA, 52 bp, synthetic construct
  misc_feature 1..52: note = synthetic
  source 1..52: mol_type = other DNA
ccaccgctgagcaataactaggagcatttcaccgtagatgagtcaacggcct

SEQ ID NO: 130 - DNA, 52 bp, synthetic construct
  misc_feature 1..52: note = synthetic
  source 1..52: mol_type = other DNA
ccaccgctgagcaataactaatgggtcttctgcgtagatgagtcaacggcct

SEQ ID NO: 131 - DNA, 52 bp, synthetic construct
  misc_feature 1..52: note = synthetic
  source 1..52: mol_type = other DNA
ccaccgctgagcaataactaaagtccgtgaaccgtagatgagtcaacggcct

SEQ ID NO: 132 - DNA, 52 bp, synthetic construct
  misc_feature 1..52: note = synthetic
  source 1..52: mol_type = other DNA
ccaccgctgagcaataactatgacatagagggcgtagatgagtcaacggcct

SEQ ID NO: 133 - DNA, 52 bp, synthetic construct
  misc_feature 1..52: note = synthetic
  source 1..52: mol_type = other DNA
ccaccgctgagcaataactacgtcaatcgtgtcgtagatgagtcaacggcct

SEQ ID NO: 134 - DNA, 52 bp, synthetic construct
  misc_feature 1..52: note = synthetic
  source 1..52: mol_type = other DNA
ccaccgctgagcaataactagttcgaagcaaccgtagatgagtcaacggcct

SEQ ID NO: 135 - DNA, 52 bp, synthetic construct
  misc_feature 1..52: note = synthetic
  source 1..52: mol_type = other DNA
ccaccgctgagcaataactaacccgaattcaccgtagatgagtcaacggcct

SEQ ID NO: 136 - DNA, 52 bp, synthetic construct
  misc_feature 1..52: note = synthetic
  source 1..52: mol_type = other DNA
ccaccgctgagcaataactagaggacttcacacgtagatgagtcaacggcct

SEQ ID NO: 137 - DNA, 52 bp, synthetic construct
  misc_feature 1..52: note = synthetic
  source 1..52: mol_type = other DNA
ccaccgctgagcaataactagattccaccgtacgtagatgagtcaacggcct

SEQ ID NO: 138 - DNA, 52 bp, synthetic construct
  misc_feature 1..52: note = synthetic
  source 1..52: mol_type = other DNA
ccaccgctgagcaataactagtattcgccatgcgtagatgagtcaacggcct

SEQ ID NO: 139 - DNA, 52 bp, synthetic construct
  misc_feature 1..52: note = synthetic
  source 1..52: mol_type = other DNA
ccaccgctgagcaataactagcttgttatccgcgtagatgagtcaacggcct

SEQ ID NO: 140 - DNA, 52 bp, synthetic construct
  misc_feature 1..52: note = synthetic
  source 1..52: mol_type = other DNA
ccaccgctgagcaataactacgtccaactatgcgtagatgagtcaacggcct

SEQ ID NO: 141 - DNA, 52 bp, synthetic construct
  misc_feature 1..52: note = synthetic
  source 1..52: mol_type = other DNA
ccaccgctgagcaataactaggtaacagtgaccgtagatgagtcaacggcct

SEQ ID NO: 142 - DNA, 52 bp, synthetic construct
  misc_feature 1..52: note = synthetic
  source 1..52: mol_type = other DNA
ccaccgctgagcaataactagcgcaaaagaagcgtagatgagtcaacggcct

SEQ ID NO: 143 - DNA, 52 bp, synthetic construct
  misc_feature 1..52: note = synthetic
  source 1..52: mol_type = other DNA
ccaccgctgagcaataactatgtggttgatcgcgtagatgagtcaacggcct

SEQ ID NO: 144 - DNA, 52 bp, synthetic construct
  misc_feature 1..52: note = synthetic
  source 1..52: mol_type = other DNA
ccaccgctgagcaataactatgtgggattgtgcgtagatgagtcaacggcct

SEQ ID NO: 145 - DNA, 52 bp, synthetic construct
  misc_feature 1..52: note = synthetic
  source 1..52: mol_type = other DNA
ccaccgctgagcaataactatgcttcgggatacgtagatgagtcaacggcct

SEQ ID NO: 146 - DNA, 52 bp, synthetic construct
  misc_feature 1..52: note = synthetic
  source 1..52: mol_type = other DNA
ccaccgctgagcaataactagacagctcgttacgtagatgagtcaacggcct

SEQ ID NO: 147 - DNA, 52 bp, synthetic construct
  misc_feature 1..52: note = synthetic
  source 1..52: mol_type = other DNA
ccaccgctgagcaataactataagaagcgctccgtagatgagtcaacggcct

SEQ ID NO: 148 - DNA, 52 bp, synthetic construct
  misc_feature 1..52: note = synthetic
  source 1..52: mol_type = other DNA
ccaccgctgagcaataactacatacacactcccgtagatgagtcaacggcct

SEQ ID NO: 149 - DNA, 52 bp, synthetic construct
  misc_feature 1..52: note = synthetic
  source 1..52: mol_type = other DNA
ccaccgctgagcaataactatgccgccaaaatcgtagatgagtcaacggcct

SEQ ID NO: 150 - DNA, 52 bp, synthetic construct
  misc_feature 1..52: note = synthetic
  source 1..52: mol_type = other DNA
ccaccgctgagcaataactacggaccttctaacgtagatgagtcaacggcct

SEQ ID NO: 151 - DNA, 52 bp, synthetic construct
  misc_feature 1..52: note = synthetic
  source 1..52: mol_type = other DNA
ccaccgctgagcaataactatctcacgtcaaccgtagatgagtcaacggcct

SEQ ID NO: 152 - DNA, 52 bp, synthetic construct
  misc_feature 1..52: note = synthetic
  source 1..52: mol_type = other DNA
ccaccgctgagcaataactacgcaagagaacacgtagatgagtcaacggcct

SEQ ID NO: 153 - DNA, 52 bp, synthetic construct
  misc_feature 1..52: note = synthetic
  source 1..52: mol_type = other DNA
ccaccgctgagcaataactattagcttccctgcgtagatgagtcaacggcct

SEQ ID NO: 154 - DNA, 52 bp, synthetic construct
  misc_feature 1..52: note = synthetic
  source 1..52: mol_type = other DNA
ccaccgctgagcaataactagaagccaagcatcgtagatgagtcaacggcct

SEQ ID NO: 155 - DNA, 52 bp, synthetic construct
  misc_feature 1..52: note = synthetic
  source 1..52: mol_type = other DNA
ccaccgctgagcaataactattcgtagcgtgtcgtagatgagtcaacggcct

SEQ ID NO: 156 - DNA, 52 bp, synthetic construct
  misc_feature 1..52: note = synthetic
  source 1..52: mol_type = other DNA
ccaccgctgagcaataactagtcgctgatcaacgtagatgagtcaacggcct

SEQ ID NO: 157 - DNA, 52 bp, synthetic construct
  misc_feature 1..52: note = synthetic
  source 1..52: mol_type = other DNA
ccaccgctgagcaataactatcaactgatcggcgtagatgagtcaacggcct

SEQ ID NO: 158 - DNA, 52 bp, synthetic construct
  misc_feature 1..52: note = synthetic
  source 1..52: mol_type = other DNA
ccaccgctgagcaataactaccagtttctacgcgtagatgagtcaacggcct

SEQ ID NO: 159 - DNA, 52 bp, synthetic construct
  misc_feature 1..52: note = synthetic
  source 1..52: mol_type = other DNA
ccaccgctgagcaataactaacccattgcgatcgtagatgagtcaacggcct

SEQ ID NO: 160 - DNA, 52 bp, synthetic construct
  misc_feature 1..52: note = synthetic
  source 1..52: mol_type = other DNA
ccaccgctgagcaataactatcaccaccctatcgtagatgagtcaacggcct

SEQ ID NO: 161 - DNA, 52 bp, synthetic construct
  misc_feature 1..52: note = synthetic
  source 1..52: mol_type = other DNA
ccaccgctgagcaataactaggtcttcacttccgtagatgagtcaacggcct

SEQ ID NO: 162 - DNA, 52 bp, synthetic construct
  misc_feature 1..52: note = synthetic
  source 1..52: mol_type = other DNA
ccaccgctgagcaataactagttagagatgggcgtagatgagtcaacggcct

SEQ ID NO: 163 - DNA, 52 bp, synthetic construct
  misc_feature 1..52: note = synthetic
  source 1..52: mol_type = other DNA
ccaccgctgagcaataactatcttgcacactccgtagatgagtcaacggcct

SEQ ID NO: 164 - DNA, 52 bp, synthetic construct
  misc_feature 1..52: note = synthetic
  source 1..52: mol_type = other DNA
ccaccgctgagcaataactattttctctgcggcgtagatgagtcaacggcct

SEQ ID NO: 165 - DNA, 52 bp, synthetic construct
  misc_feature 1..52: note = synthetic
  source 1..52: mol_type = other DNA
ccaccgctgagcaataactatcagccgagttacgtagatgagtcaacggcct

SEQ ID NO: 166 - DNA, 52 bp, synthetic construct
  misc_feature 1..52: note = synthetic
  source 1..52: mol_type = other DNA
ccaccgctgagcaataactactcgtgatcagacgtagatgagtcaacggcct

SEQ ID NO: 167 - DNA, 52 bp, synthetic construct
  misc_feature 1..52: note = synthetic
  source 1..52: mol_type = other DNA
ccaccgctgagcaataactacctttctcggaacgtagatgagtcaacggcct

SEQ ID NO: 168 - DNA, 52 bp, synthetic construct
  misc_feature 1..52: note = synthetic
  source 1..52: mol_type = other DNA
ccaccgctgagcaataactaacgctagagcttcgtagatgagtcaacggcct

SEQ ID NO: 169 - DNA, 52 bp, synthetic construct
  misc_feature 1..52: note = synthetic
  source 1..52: mol_type = other DNA
ccaccgctgagcaataactattccccgtttagcgtagatgagtcaacggcct

SEQ ID NO: 170 - DNA, 52 bp, synthetic construct
  misc_feature 1..52: note = synthetic
  source 1..52: mol_type = other DNA
ccaccgctgagcaataactaagaatcgcaacccgtagatgagtcaacggcct

SEQ ID NO: 171 - DNA, 52 bp, synthetic construct
  misc_feature 1..52: note = synthetic
  source 1..52: mol_type = other DNA
ccaccgctgagcaataactaggaaggaactgtcgtagatgagtcaacggcct

SEQ ID NO: 172 - DNA, 52 bp, synthetic construct
  misc_feature 1..52: note = synthetic
  source 1..52: mol_type = other DNA
ccaccgctgagcaataactacttggcatcttccgtagatgagtcaacggcct

SEQ ID NO: 173 - DNA, 52 bp, synthetic construct
  misc_feature 1..52: note = synthetic
  source 1..52: mol_type = other DNA
ccaccgctgagcaataactaaggccgatttgtcgtagatgagtcaacggcct

SEQ ID NO: 174 - DNA, 52 bp, synthetic construct
  misc_feature 1..52: note = synthetic
  source 1..52: mol_type = other DNA
ccaccgctgagcaataactaaacaaagggtcccgtagatgagtcaacggcct

SEQ ID NO: 175 - DNA, 52 bp, synthetic construct
  misc_feature 1..52: note = synthetic
  source 1..52: mol_type = other DNA
ccaccgctgagcaataactacaattggtagcccgtagatgagtcaacggcct

SEQ ID NO: 176 - DNA, 52 bp, synthetic construct
  misc_feature 1..52: note = synthetic
  source 1..52: mol_type = other DNA
ccaccgctgagcaataactaaccatcgactcacgtagatgagtcaacggcct

SEQ ID NO: 177 - DNA, 52 bp, synthetic construct
  misc_feature 1..52: note = synthetic
  source 1..52: mol_type = other DNA
ccaccgctgagcaataactacgtgagatgaaccgtagatgagtcaacggcct

SEQ ID NO: 178 - DNA, 52 bp, synthetic construct
  misc_feature 1..52: note = synthetic
  source 1..52: mol_type = other DNA
ccaccgctgagcaataactaccatggtcttgtcgtagatgagtcaacggcct

SEQ ID NO: 179 - DNA, 52 bp, synthetic construct
  misc_feature 1..52: note = synthetic
  source 1..52: mol_type = other DNA
ccaccgctgagcaataactaagatatgagcgccgtagatgagtcaacggcct

SEQ ID NO: 180 - DNA, 52 bp, synthetic construct
  misc_feature 1..52: note = synthetic
  source 1..52: mol_type = other DNA
ccaccgctgagcaataactagtgtgacagagtcgtagatgagtcaacggcct

SEQ ID NO: 181 - DNA, 52 bp, synthetic construct
  misc_feature 1..52: note = synthetic
  source 1..52: mol_type = other DNA
ccaccgctgagcaataactaattgtgtgacggcgtagatgagtcaacggcct

SEQ ID NO: 182 - DNA, 52 bp, synthetic construct
  misc_feature 1..52: note = synthetic
  source 1..52: mol_type = other DNA
ccaccgctgagcaataactacggtagtttgctcgtagatgagtcaacggcct

SEQ ID NO: 183 - DNA, 52 bp, synthetic construct
  misc_feature 1..52: note = synthetic
  source 1..52: mol_type = other DNA
ccaccgctgagcaataactaggacatgtccatcgtagatgagtcaacggcct

SEQ ID NO: 184 - DNA, 52 bp, synthetic construct
  misc_feature 1..52: note = synthetic
  source 1..52: mol_type = other DNA
ccaccgctgagcaataactattgagggagacacgtagatgagtcaacggcct

SEQ ID NO: 185 - DNA, 52 bp, synthetic construct
  misc_feature 1..52: note = synthetic
  source 1..52: mol_type = other DNA
ccaccgctgagcaataactacgacatcctctacgtagatgagtcaacggcct

SEQ ID NO: 186 - DNA, 52 bp, synthetic construct
  misc_feature 1..52: note = synthetic
  source 1..52: mol_type = other DNA
ccaccgctgagcaataactatgagcgagttcacgtagatgagtcaacggcct

SEQ ID NO: 187 - DNA, 52 bp, synthetic construct
  misc_feature 1..52: note = synthetic
  source 1..52: mol_type = other DNA
ccaccgctgagcaataactagaccttcggattcgtagatgagtcaacggcct

SEQ ID NO: 188 - DNA, 52 bp, synthetic construct
  misc_feature 1..52: note = synthetic
  source 1..52: mol_type = other DNA
ccaccgctgagcaataactatgtagatccgcacgtagatgagtcaacggcct

SEQ ID NO: 189 - DNA, 52 bp, synthetic construct
  misc_feature 1..52: note = synthetic
  source 1..52: mol_type = other DNA
ccaccgctgagcaataactatggcactctagacgtagatgagtcaacggcct

SEQ ID NO: 190 - DNA, 52 bp, synthetic construct
  misc_feature 1..52: note = synthetic
  source 1..52: mol_type = other DNA
ccaccgctgagcaataactaaacagtagtcggcgtagatgagtcaacggcct

SEQ ID NO: 191 - DNA, 52 bp, synthetic construct
  misc_feature 1..52: note = synthetic
  source 1..52: mol_type = other DNA
ccaccgctgagcaataactatcatgcggaaagcgtagatgagtcaacggcct

SEQ ID NO: 192 - DNA, 52 bp, synthetic construct
  misc_feature 1..52: note = synthetic
  source 1..52: mol_type = other DNA
ccaccgctgagcaataactatcgaatcgtgtccgtagatgagtcaacggcct

SEQ ID NO: 193 - DNA, 52 bp, synthetic construct
  misc_feature 1..52: note = synthetic
  source 1..52: mol_type = other DNA
ccaccgctgagcaataactaggtgtatagccacgtagatgagtcaacggcct

SEQ ID NO: 194 - DNA, 52 bp, synthetic construct
  misc_feature 1..52: note = synthetic
  source 1..52: mol_type = other DNA
ccaccgctgagcaataactattgcagtgcaagcgtagatgagtcaacggcct

SEQ ID NO: 195 - DNA, 52 bp, synthetic construct
  misc_feature 1..52: note = synthetic
  source 1..52: mol_type = other DNA
ccaccgctgagcaataactacgattgcagaagcgtagatgagtcaacggcct

SEQ ID NO: 196 - DNA, 52 bp, synthetic construct
  misc_feature 1..52: note = synthetic
  source 1..52: mol_type = other DNA
ccaccgctgagcaataactaccagacgttgttcgtagatgagtcaacggcct

SEQ ID NO: 197 - DNA, 52 bp, synthetic construct
  misc_feature 1..52: note = synthetic
  source 1..52: mol_type = other DNA
ccaccgctgagcaataactatggtggccataacgtagatgagtcaacggcct

SEQ ID NO: 198 - DNA, 52 bp, synthetic construct
  misc_feature 1..52: note = synthetic
  source 1..52: mol_type = other DNA
ccaccgctgagcaataactacagagtcaatggcgtagatgagtcaacggcct

SEQ ID NO: 199 - DNA, 52 bp, synthetic construct
  misc_feature 1..52: note = synthetic
  source 1..52: mol_type = other DNA
ccaccgctgagcaataactacctatcattccccgtagatgagtcaacggcct

SEQ ID NO: 200 - DNA, 52 bp, synthetic construct
  misc_feature 1..52: note = synthetic
  source 1..52: mol_type = other DNA
ccaccgctgagcaataactagaggtatgactccgtagatgagtcaacggcct

SEQ ID NO: 201 - DNA, 52 bp, synthetic construct
  misc_feature 1..52: note = synthetic
  source 1..52: mol_type = other DNA
ccaccgctgagcaataactactaggtcaagtccgtagatgagtcaacggcct

SEQ ID NO: 202 - DNA, 52 bp, synthetic construct
  misc_feature 1..52: note = synthetic
  source 1..52: mol_type = other DNA
ccaccgctgagcaataactaactcggctttcacgtagatgagtcaacggcct

SEQ ID NO: 203 - DNA, 52 bp, synthetic construct
  misc_feature 1..52: note = synthetic
  source 1..52: mol_type = other DNA
ccaccgctgagcaataactattcacaagcggacgtagatgagtcaacggcct

(19) United States
(12) Patent Application Publication
(10) Pub. No.: US 2024/0309041 A1
(43) Pub. Date: Sep. 19, 2024

(54) METHODS FOR TARGETED PROTEIN QUANTIFICATION BY BAR-CODING AFFINITY REAGENT WITH UNIQUE DNA SEQUENCES

(71) Applicant: ARIZONA BOARD OF REGENTS ON BEHALF OF ARIZONA STATE UNIVERSITY, Scottsdale, AZ (US)

(72) Inventor: Joshua LaBaer, Chandler, AZ (US)

(21) Appl. No.: 18/675,970

(22) Filed: May 28, 2024

Related U.S. Application Data
(63) Continuation of application No. 16/811,573, filed on Mar. 6, 2020, now Pat. No. 12,030,909.
Application No. 16/811,573 is a continuation of application No. 16/480,601, filed on Jul. 24, 2019, now Pat. No. 10,618,932.
(60) Provisional application No. 62/461,681, filed on Feb. 21, 2017.

(51) Int. Cl. C07H 21/04; B01D 15/38; G01N 33/68

(52) U.S. Cl. CPC C07H 21/04; B01D 15/3819; G01N 33/6803; G01N 33/6854; G01N 2458/10

(57) ABSTRACT

Provided herein are affinity reagents having affinity for particular target, each reagent having a unique DNA barcode, and methods for using the same to measure the abundance of targets in a sample. In particular, methods are provided in which unique barcodes linked to affinity reagents are contacted to a sample to bind antigens if present in said sample. In cases in which the affinity reagents are antibodies and the targets are antigens, antibodies that are bound to their target antigens can be separated from unbound antibodies and the DNA barcode associated with the affinity reagent is amplified, such as with a PCR reaction. In some cases, amplified barcode DNA is subjected to DNA sequencing as a measure of the levels of the target protein in the sample.

### CROSS-REFERENCE TO RELATED APPLICATIONS

This application is a continuation of U.S. patent application Ser. No. 16/811,573, filed Mar. 6, 2020, which is a continuation of U.S. patent application Ser. No. 16/480,601, filed Jul. 24, 2019, which represents the national stage entry of PCT International Application No. PCT/US2018/018908, filed on Feb. 21, 2018, and claims the benefit of U.S. Application Ser. No. 62/461,681, filed on Feb. 21, 2017, each of which is incorporated by reference herein as if set forth in its entirety.

### REFERENCE TO AN ELECTRONIC SEQUENCE LISTING

The contents of the electronic sequence listing (112624.01454.xml: Size: 258,150 bytes: and Date of Creation: May 24, 2024) is herein incorporated by reference in its entirety.

### STATEMENT REGARDING FEDERALLY SPONSORED RESEARCH OR DEVELOPMENT Not applicable.

### BACKGROUND

With the advent of various ‘omics’ technologies and methods which stratify samples and diseases based on measuring many variables simultaneously, there is an increasing demand for high throughput tools that quantify specific targets. There are already numerous genomics tools that assess gene expression, gene copy number, mutations, etc. at a global scale to determine subtypes of disease that might be useful for prognostication and management of therapy. But it is well known that the genome (which is a blue print) does not always reflect the actual state of biology at any time and gene measurements are not always possible from readily accessible samples like blood. Thus, there is a strong desire to have similar high throughput tools to measure the proteome, which is the product of the genome and more closely reflects the current state of biology. However, high throughput measurement of the proteome is much more challenging than similar genome measurements, because there is no protein equivalent to the base pairing measurements that emerge from the inherent double-stranded nature of DNA.

There is a wide variety of methods to measure proteins. These can be generally divided into antibody-based methods and chemistry-based methods. By far, the most common chemistry-based method is mass spectrometry, which is most commonly employed by ionizing peptides (created by proteolytic digestion) and measuring their mobility in a magnetic field. The accuracy of these instruments is sufficient to identify virtually any protein by comparing its spectrum to spectrums predicted from the genome. Although nearly universal in its ability to detect proteins and even modified proteins, mass spectrometry is very low throughput. A thorough examination of single sample can take hours and it requires great care to run a set samples in a fashion that allows comparison of one run to the next. There are many other tools that detect proteins chemically, but they are not capable of identifying specific proteins in a universal manner.

Detection of proteins is most commonly accomplished with antibodies (or more generally, affinity reagents), and include many different configurations such as western blots, immunoprecipitation, flow cytometry, reverse phase protein arrays, enzyme linked immunosorbent assay (ELISA), and many others. These applications all rely on antibodies that recognize specific targets, and which can bind with extraordinary selectivity and affinity. There are currently more than 2,000,000 antibodies available on the market that target a large fraction of the human proteome. It is important to note that not all antibodies are high quality, but many are quite good and methods to produce antibodies have become routine. Although the use of an antibody to measure its target can be relatively fast, it is not straightforward to multiplex measurements using many antibodies simultaneously. Accordingly, there remains a need in the art for improved methods for simultaneous multiplexed detection and measurement of many proteins (including specific post-translational forms of proteins) or other target molecules.

### SUMMARY

In a first aspect, provided herein is a composition comprising a plurality of modified affinity reagents, each affinity reagent of the plurality comprising a unique identifying nucleotide sequence relative to other affinity reagents of the plurality, wherein each identifying nucleotide sequence is flanked by a first amplifying nucleotide sequence and a second amplifying nucleotide sequence. Affinity reagents of the plurality can be antibodies. Affinity reagents of the plurality can be peptide aptamers or nucleic acid aptamers. An identifying nucleotide sequence can be attached to an affinity reagent by a linker comprising a cleavable protein photocrosslinker. An identifying nucleotide sequence can be attached to an affinity reagent by a linker comprising a fluorescent moiety. Unique identifying nucleotide sequences of the plurality can comprise one or more of SEQ ID Nos: 104-203.

In another aspect, provided herein is a method for high throughput target molecule identification and quantification. The method can comprise or consist essentially of contacting a sample with a modified affinity reagent under conditions that promote binding of the modified affinity reagent to its target molecule if present in the contacted sample; removing unbound modified affinity reagent from the contacted sample; and amplifying and sequencing an identifying nucleotide sequence coupled to said modified affinity reagent whereby the target molecule is identified and quantified based on detection of the identifying nucleotide sequence. The method can further comprise adding a linker to an affinity reagent to form the modified affinity reagent, wherein the linker comprises the identifying nucleotide sequence flanked by a pair of amplifying nucleotide sequences. The affinity reagent can be an antibody. The adding step can further comprise adding a linker to a region of the antibody that is not an antigen binding region. The adding step can further comprise adding a linker to a fragment crystallizable region (Fc region) of the antibody. The affinity reagent can be an aptamer. The identifying nucleotide sequence can have a length of about 10 nucleotides to about 20 nucleotides. The identifying nucleotide sequence can have a length of about 12 nucleotides. The linker can be selected from SEQ ID Nos: 104-203. The identifying nucleotide sequence can comprise SEQ ID NO:1 or a barcode sequence set forth in Table 1. The identifying nucleotide sequence can comprise about 50% of AT base pairs and about 50% of GC base pairs. The amplifying sequence can have a length ranging from 20 to 30 base pair. The amplifying sequence can comprise SEQ ID NO:2. The amplifying sequence can comprise SEQ ID NO:3. The linker can further comprise a fluorescent protein or a cleavable protein photocrosslinker.

In a further aspect, provided herein is a kit for high throughput protein quantification. The kit can comprise X modified affinity reagent(s), where X is equal to or greater than 1, each modified affinity reagent comprising a linker, where the linker comprising an identifying nucleotide sequence flanked by a pair of amplifying nucleotide sequences; and each modified affinity reagent comprising a different identifying nucleotide sequence from other modified affinity reagents. The linker can be selected from SEQ ID Nos: 104-203.

### BRIEF DESCRIPTION OF THE DRAWINGS

The present disclosure will be better understood and features, aspects, and advantages other than those set forth above will become apparent when consideration is given to the following detailed description thereof. Such detailed description makes reference to the following drawings, wherein:

FIG. 1 is a flowchart that illustrates steps of a method for high throughput protein quantification.

FIG. 2 demonstrates cloning and protein expression of the HPV proteome. An SDS-PAGE gel shows the expression of antigens of different HPV subtypes. (*) indicates an unexpressed protein. Further experiments confirmed expression of HPV58 E7, L1, and L2.

FIG. 3 demonstrates immune response screening of OPC patient sera. Percent barcode enrichment of control and OPC patient sera after barcode amplification and next generation sequencing.

FIG. 4 is an image of a DNA gel showing the enrichment of antibodies against E1 and E7 antigens in OPC patient P3 with barcode specific primers.

### DETAILED DESCRIPTION

The compositions and methods described herein couples the ability of antibodies (or virtually any affinity reagent) to recognize their targets with a unique DNA barcode that enables the detection of the antibody using, for example, next generation DNA sequencing methods. This disclosure is based at least in part on the inventor's development of a quantitative, multiplexed, bar-coded antigen library for detection and measurement of immune responses in pathogen-induced cancers including, for example, multiple serotypes of HPV (Human Papillomavirus)-positive Oropharyngeal carcinomas (OPC).

### Affinity Reagents

Accordingly, in a first aspect, provided herein are affinity reagents having affinity for particular target molecules and comprising a unique DNA barcode, where the affinity reagent is useful to detect and measure the abundance of targets in a sample. Advantageously, a plurality of affinity reagents can be used to simultaneously measure a plurality of targets in a single sample. Accordingly, in some cases, affinity reagents of this disclosure are provided as a library of affinity reagents for multiplexed detection and measurement of multiple distinct targets in a single sample. As used herein, the term “affinity reagent” refers to an antibody, peptide, nucleic acid, or other small molecule that specifically binds to a biological molecule (“biomolecule”) of interest in order to identify, track, capture, and/or influence its activity. In some embodiments, the affinity reagent is an antibody. In other embodiments, the affinity reagent is an aptamer.

In some cases, the affinity reagents are antibodies having specificity for particular protein (e.g., antigen) targets, where the antibodies are linked to a DNA barcode. In such cases, an antibody affinity reagent is contacted to a sample under conditions that promote binding of the affinity reagent to its target antigen when present in said sample. Antibodies that are bound to their target antigens can be separated from unbound antibodies by washing unbound reagents from the sample. In some embodiments, the DNA bar code associated with the affinity reagent is amplified, such as by polymerase chain reaction (PCR), and the amplified barcode DNA is subjected to DNA sequencing to provide a measure of target antigen in the contacted sample.

Any antibody can be used for the affinity reagents of this disclosure. Preferably, the antibodies bind tightly (i.e., have high affinity for) target antigens. It will be understood that antibodies selected for use in affinity reagents will vary according to the particular application. In some cases, the antibodies have affinity for a particular protein only when in a certain conformation or having a specific modification.

In some embodiments, one or more modifications are made to the fragment crystallizable region (Fc region) of the affinity reagent antibody. The Fc region is the tail region of an antibody that interacts with cell surface receptors and some proteins of the complement system. In other embodiments, the modification is made to a common region far from the target binding region. In this manner, one may obtain a library of antibodies affinity reagents having specificity for desired targets, each antibody chemically modified to include a linked DNA barcode of known sequence. In certain embodiments, the DNA barcode sequence is flanked by common sequences.

In other embodiments, the affinity reagents are aptamers. Aptamers are peptides and nucleic acid molecules that bind specifically to various biological molecules and are useful for in vitro or in vivo localization and quantification of various biological molecules. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Generally, nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are peptides selected or engineered to bind specific target molecules. These proteins consist of one or more peptide loops of variable sequence displayed by a protein scaffold. They can be isolated from combinatorial libraries and, in some cases, modified by directed mutation or rounds of variable region mutagenesis and selection. In vivo, peptide aptamers can bind cellular protein targets and exert biological effects, including interference with the normal protein interactions of their targeted molecules with other proteins. Libraries of peptide aptamers have been used as “mutagens,” in studies in which an investigator introduces a library that expresses different peptide aptamers into a cell population, selects for a desired phenotype, and identifies those aptamers associated with that phenotype.

As demonstrated in the Example section herein, genes from multiple HPV strains were cloned and expressed in vitro to produce a library of HPV antigens. When DNA barcodes and their flanking sequences were linked to these antigens, the resulting affinity reagents could detect the presence of particular HPV strain DNA in patient samples.

Like antibody affinity reagents, aptamer affinity reagents comprise a linked DNA barcode sequence. In some cases, the linker is a cleavable protein photocrosslinker, which can be photo-cleaved from the antibody or aptamer. In other cases, the linker is a ligand comprising a DNA barcode which can append to a target with a fusion tag. For example, the linker may be a Halo ligand comprising a barcode sequence appended to a Halo fusion tag. In other cases, the linker comprises a fluorescent probe in addition to the DNA barcode.

Once the library of antibodies is assembled. Each antibody is chemically modified in step 140 to add a linker that includes a unique DNA barcode, which is an identifying sequence flanked at its 5′ and 3′ ends by a set of common sequences. In certain embodiments, the DNA barcode comprises a nucleotide sequence of GCTGTACGGATT (SEQ ID NO:1). Other DNA barcode sequences are set forth in Table 1. Exemplary linker sequences are set forth in Table 2. The common sequences act as a pair of amplifying sequences. In some embodiments, each barcode sequence (bold font) is flanked by a 5′ flanking sequence and a 3′ flanking sequence. In some cases, the 5′ flanking sequence is (CCACCGCTGAGCAATAACTA; SEQ ID NO:2). In some cases, the 3′ flanking sequence is (CGTAGATGAGTCAACGGCCT; SEQ ID NO:3).

TABLE 1
Exemplary Barcode Sequences

| | | Barcode |
|---|---|---|
| Barcode name | barcode sequence | SEQ ID NO: |
| Halo_BC1 | GTAGTGACAGGT | 4 |
| Halo_BC2 | TCTGTGAAGTCC | 5 |
| Halo_BC3 | ATCAGATCGCCT | 6 |
| Halo_BC4 | AATGTGGTCTCG | 7 |
| Halo_BC5 | CCTCTCCAAACA | 8 |
| Halo_BC6 | TACTGGACAAGG | 9 |
| Halo_BC7 | TATCGGAGTCCT | 10 |
| Halo_BC8 | GGTGGAGTTACT | 11 |
| Halo_BC9 | CGGCTACTATTG | 12 |
| Halo_BC10 | CCGAGCTATGTA | 13 |
| Halo_BC11 | ACTACGTCCAAC | 14 |
| Halo_BC12 | TTCATCCGAACG | 15 |
| Halo_BC13 | CGAAACGCTTAG | 16 |
| Halo_BC14 | GCCTAAGTTCCA | 17 |
| Halo_BC15 | CAATTCCCACGT | 18 |
| Halo_BC16 | CGGTGAGACATA | 19 |
| Halo_BC17 | CTCTGAGGTTTG | 20 |
| Halo_BC18 | TACTGTCACCCA | 21 |
| Halo_BC19 | CAGGAGGTACAT | 22 |
| Halo_BC20 | CTTCCTACAGCA | 23 |
| Halo_BC21 | TAGAAACCGAGG | 24 |
| Halo_BC22 | GAAAAGCGTACC | 25 |
| Halo_BC23 | CGCTCATAACTC | 26 |
| Halo_BC24 | GGCATATACGAC | 27 |
| Halo_BC25 | GTGCTCTATCAC | 28 |
| Halo_BC26 | GGAGCATTTCAC | 29 |
| Halo_BC27 | ATGGGTCTTCTG | 30 |
| Halo_BC28 | AAGTCCGTGAAC | 31 |
| Halo_BC29 | TGACATAGAGGG | 32 |
| Halo_BC30 | CGTCAATCGTGT | 33 |
| Halo_BC31 | GTTCGAAGCAAC | 34 |
| Halo_BC32 | ACCCGAATTCAC | 35 |
| Halo_BC33 | GAGGACTTCACA | 36 |
| Halo_BC34 | GATTCCACCGTA | 37 |
| Halo_BC35 | GTATTCGCCATG | 38 |
| Halo_BC36 | GCTTGTTATCCG | 39 |
| Halo_BC37 | CGTCCAACTATG | 40 |
| Halo_BC38 | GGTAACAGTGAC | 41 |
| Halo_BC39 | GCGCAAAAGAAG | 42 |
| Halo_BC40 | TGTGGTTGATCG | 43 |
| Halo_BC41 | TGTGGGATTGTG | 44 |
| Halo_BC42 | TGCTTCGGGATA | 45 |
| Halo_BC43 | GACAGCTCGTTA | 46 |
| Halo_BC44 | TAAGAAGCGCTC | 47 |
| Halo_BC45 | CATACACACTCC | 48 |
| Halo_BC46 | TGCCGCCAAAAT | 49 |
| Halo_BC47 | CGGACCTTCTAA | 50 |
| Halo_BC48 | TCTCACGTCAAC | 51 |
| Halo_BC49 | CGCAAGAGAACA | 52 |
| Halo_BC50 | TTAGCTTCCCTG | 53 |
| Halo_BC51 | GAAGCCAAGCAT | 54 |
| Halo_BC52 | TTCGTAGCGTGT | 55 |
| Halo_BC53 | GTCGCTGATCAA | 56 |
| Halo_BC54 | TCAACTGATCGG | 57 |
| Halo_BC55 | CCAGTTTCTACG | 58 |
| Halo_BC56 | ACCCATTGCGAT | 59 |
| Halo_BC57 | TCACCACCCTAT | 60 |
| Halo_BC58 | GGTCTTCACTTC | 61 |
| Halo_BC59 | GTTAGAGATGGG | 62 |
| Halo_BC60 | TCTTGCACACTC | 63 |
| Halo_BC61 | TTTTCTCTGCGG | 64 |
| Halo_BC62 | TCAGCCGAGTTA | 65 |
| Halo_BC63 | CTCGTGATCAGA | 66 |
| Halo_BC64 | CCTTTCTCGGAA | 67 |
| Halo_BC65 | ACGCTAGAGCTT | 68 |
| Halo_BC66 | TTCCCCGTTTAG | 69 |
| Halo_BC67 | AGAATCGCAACC | 70 |
| Halo_BC68 | GGAAGGAACTGT | 71 |
| Halo_BC69 | CTTGGCATCTTC | 72 |
| Halo_BC70 | AGGCCGATTTGT | 73 |
| Halo_BC71 | AACAAAGGGTCC | 74 |
| Halo_BC72 | CAATTGGTAGCC | 75 |
| Halo_BC73 | ACCATCGACTCA | 76 |
| Halo_BC74 | CGTGAGATGAAC | 77 |
| Halo_BC75 | CCATGGTCTTGT | 78 |
| Halo_BC76 | CAGATATGAGCGC | 79 |
| Halo_BC77 | GTGTGACAGAGT | 80 |
| Halo_BC78 | ATTGTGTGACGG | 81 |
| Halo_BC79 | CGGTAGTTTGCT | 82 |
| Halo_BC80 | GGACATGTCCAT | 83 |
| Halo_BC81 | TTGAGGGAGACA | 84 |
| Halo_BC82 | CGACATCCTCTA | 85 |
| Halo_BC83 | TGAGCGAGTTCA | 86 |
| Halo_BC84 | GACCTTCGGATT | 87 |
| Halo_BC85 | TGTAGATCCGCA | 88 |
| Halo_BC86 | TGGCACTCTAGA | 89 |
| Halo_BC87 | AACAGTAGTCGG | 90 |
| Halo_BC88 | TCATGCGGAAAG | 91 |
| Halo_BC89 | TCGAATCGTGTC | 92 |
| Halo_BC90 | GGTGTATAGCCA | 93 |
| Halo_BC91 | TTGCAGTGCAAG | 94 |
| Halo_BC92 | CGATTGCAGAAG | 95 |
| Halo_BC93 | CCAGACGTTGTT | 96 |
| Halo_BC94 | TGGTGGCCATAA | 97 |
| Halo_BC95 | CAGAGTCAATGG | 98 |
| Halo_BC96 | CCTATCATTCCC | 99 |
| Halo_BC97 | GAGGTATGACTC | 100 |
| Halo_BC98 | CTAGGTCAAGTC | 101 |
| Halo_BC99 | ACTCGGCTTTCA | 102 |
| Halo_BC100 | TTCACAAGCGGA | 103 |

TABLE 2
Exemplary Linker Sequences

| Name of | | |
|---|---|---|
| barcode | Linker: | SEQ |
| included | flanking seq - barcode sequence - | ID |
| in linker | flanking seq | NO: |
| Halo_BC1 | CCACCGCTGAGCAATAACTA GTAGTGACAGGT | 104 |
| | CGTAGATGAGTCAACGGCCT | |
| Halo_BC2 | CCACCGCTGAGCAATAACTA TCTGTGAAGTCC | 105 |
| | CGTAGATGAGTCAACGGCCT | |
| Halo_BC3 | CCACCGCTGAGCAATAACTA ATCAGATCGCCT | 106 |
| | CGTAGATGAGTCAACGGCCT | |
| Halo_BC4 | CCACCGCTGAGCAATAACTA AATGTGGTCTCG | 107 |
| | CGTAGATGAGTCAACGGCCT | |
| Halo_BC5 | CCACCGCTGAGCAATAACTA CCTCTCCAAACA | 108 |
| | CGTAGATGAGTCAACGGCCT | |
| Halo_BC6 | CCACCGCTGAGCAATAACTA TACTGGACAAGG | 109 |
| | CGTAGATGAGTCAACGGCCT | |
| Halo_BC7 | CCACCGCTGAGCAATAACTA TATCGGAGTCCT | 110 |
| | CGTAGATGAGTCAACGGCCT | |
| Halo_BC8 | CCACCGCTGAGCAATAACTA GGTGGAGTTACT | 111 |
| | CGTAGATGAGTCAACGGCCT | |
| Halo_BC9 | CCACCGCTGAGCAATAACTA CGGCTACTATTG | 112 |
| | CGTAGATGAGTCAACGGCCT | |
| Halo_BC10 | CCACCGCTGAGCAATAACTA CCGAGCTATGTA | 113 |
| | CGTAGATGAGTCAACGGCCT | |
| Halo_BC11 | CCACCGCTGAGCAATAACTA ACTACGTCCAAC | 114 |
| | CGTAGATGAGTCAACGGCCT | |
| Halo_BC12 | CCACCGCTGAGCAATAACTA TTCATCCGAACG | 115 |
| | CGTAGATGAGTCAACGGCCT | |
| Halo_BC13 | CCACCGCTGAGCAATAACTA CGAAACGCTTAG | 116 |
| | CGTAGATGAGTCAACGGCCT | |
| Halo_BC14 | CCACCGCTGAGCAATAACTA GCCTAAGTTCCA | 117 |
| | CGTAGATGAGTCAACGGCCT | |
| Halo_BC15 | CCACCGCTGAGCAATAACTA CAATTCCCACGT | 118 |
| | CGTAGATGAGTCAACGGCCT | |
| Halo_BC16 | CCACCGCTGAGCAATAACTA CGGTGAGACATA | 119 |
| | CGTAGATGAGTCAACGGCCT | |
| Halo_BC17 | CCACCGCTGAGCAATAACTA CTCTGAGGTTTG | 120 |
| | CGTAGATGAGTCAACGGCCT | |
| Halo_BC18 | CCACCGCTGAGCAATAACTA TACTGTCACCCA | 121 |
| | CGTAGATGAGTCAACGGCCT | |
| Halo_BC19 | CCACCGCTGAGCAATAACTA CAGGAGGTACAT | 122 |
| | CGTAGATGAGTCAACGGCCT | |
| Halo_BC20 | CCACCGCTGAGCAATAACTA CTTCCTACAGCA | 123 |
| | CGTAGATGAGTCAACGGCCT | |
| Halo_BC21 | CCACCGCTGAGCAATAACTA TAGAAACCGAGG | 124 |
| | CGTAGATGAGTCAACGGCCT | |
| Halo_BC22 | CCACCGCTGAGCAATAACTA GAAAAGCGTACC | 125 |
| | CGTAGATGAGTCAACGGCCT | |
| Halo_BC23 | CCACCGCTGAGCAATAACTA CGCTCATAACTC | 126 |
| | CGTAGATGAGTCAACGGCCT | |
| Halo_BC24 | CCACCGCTGAGCAATAACTA GGCATATACGAC | 127 |
| | CGTAGATGAGTCAACGGCCT | |
| Halo_BC25 | CCACCGCTGAGCAATAACTA GTGCTCTATCAC | 128 |
| | CGTAGATGAGTCAACGGCCT | |
| Halo_BC26 | CCACCGCTGAGCAATAACTA GGAGCATTTCAC | 129 |
| | CGTAGATGAGTCAACGGCCT | |
| Halo_BC27 | CCACCGCTGAGCAATAACTA ATGGGTCTTCTG | 130 |
| | CGTAGATGAGTCAACGGCCT | |
| Halo_BC28 | CCACCGCTGAGCAATAACTA AAGTCCGTGAAC | 131 |
| | CGTAGATGAGTCAACGGCCT | |
| Halo_BC29 | CCACCGCTGAGCAATAACTA TGACATAGAGGG | 132 |
| | CGTAGATGAGTCAACGGCCT | |
| Halo_BC30 | CCACCGCTGAGCAATAACTA CGTCAATCGTGT | 133 |
| | CGTAGATGAGTCAACGGCCT | |
| Halo_BC31 | CCACCGCTGAGCAATAACTA GTTCGAAGCAAC | 134 |
| | CGTAGATGAGTCAACGGCCT | |
| Halo_BC32 | CCACCGCTGAGCAATAACTA ACCCGAATTCAC | 135 |
| | CGTAGATGAGTCAACGGCCT | |
| Halo_BC33 | CCACCGCTGAGCAATAACTA GAGGACTTCACA | 136 |
| | CGTAGATGAGTCAACGGCCT | |
| Halo_BC34 | CCACCGCTGAGCAATAACTA GATTCCACCGTA | 137 |
| | CGTAGATGAGTCAACGGCCT | |
| Halo_BC35 | CCACCGCTGAGCAATAACTA GTATTCGCCATG | 138 |
| | CGTAGATGAGTCAACGGCCT | |
| Halo_BC36 | CCACCGCTGAGCAATAACTA GCTTGTTATCCG | 139 |
| | CGTAGATGAGTCAACGGCCT | |
| Halo_BC37 | CCACCGCTGAGCAATAACTA CGTCCAACTATG | 140 |
| | CGTAGATGAGTCAACGGCCT | |
| Halo_BC38 | CCACCGCTGAGCAATAACTA GGTAACAGTGAC | 141 |
| | CGTAGATGAGTCAACGGCCT | |
| Halo_BC39 | CCACCGCTGAGCAATAACTA GCGCAAAAGAAG | 142 |
| | CGTAGATGAGTCAACGGCCT | |
| Halo_BC40 | CCACCGCTGAGCAATAACTA TGTGGTTGATCG | 143 |
| | CGTAGATGAGTCAACGGCCT | |
| Halo_BC41 | CCACCGCTGAGCAATAACTA TGTGGGATTGTG | 144 |
| | CGTAGATGAGTCAACGGCCT | |
| Halo_BC42 | CCACCGCTGAGCAATAACTA TGCTTCGGGATA | 145 |
| | CGTAGATGAGTCAACGGCCT | |
| Halo_BC43 | CCACCGCTGAGCAATAACTA GACAGCTCGTTA | 146 |
| | CGTAGATGAGTCAACGGCCT | |
| Halo_BC44 | CCACCGCTGAGCAATAACTA TAAGAAGCGCTC | 147 |
| | CGTAGATGAGTCAACGGCCT | |
| Halo_BC45 | CCACCGCTGAGCAATAACTA CATACACACTCC | 148 |
| | CGTAGATGAGTCAACGGCCT | |
| Halo_BC46 | CCACCGCTGAGCAATAACTA TGCCGCCAAAAT | 149 |
| | CGTAGATGAGTCAACGGCCT | |
| Halo_BC47 | CCACCGCTGAGCAATAACTA CGGACCTTCTAA | 150 |
| | CGTAGATGAGTCAACGGCCT | |
| Halo_BC48 | CCACCGCTGAGCAATAACTA TCTCACGTCAAC | 151 |
| | CGTAGATGAGTCAACGGCCT | |
| Halo_BC49 | CCACCGCTGAGCAATAACTA CGCAAGAGAACA | 152 |
| | CGTAGATGAGTCAACGGCCT | |
| Halo_BC50 | CCACCGCTGAGCAATAACTA TTAGCTTCCCTG | 153 |
| | CGTAGATGAGTCAACGGCCT | |
| Halo_BC51 | CCACCGCTGAGCAATAACTA GAAGCCAAGCAT | 154 |
| | CGTAGATGAGTCAACGGCCT | |
| Halo_BC52 | CCACCGCTGAGCAATAACTA TTCGTAGCGTGT | 155 |
| | CGTAGATGAGTCAACGGCCT | |
| Halo_BC53 | CCACCGCTGAGCAATAACTA GTCGCTGATCAA | 156 |
| | CGTAGATGAGTCAACGGCCT | |
| Halo_BC54 | CCACCGCTGAGCAATAACTA TCAACTGATCGG | 157 |
| | CGTAGATGAGTCAACGGCCT | |
| Halo_BC55 | CCACCGCTGAGCAATAACTA CCAGTTTCTACG | 158 |
| | CGTAGATGAGTCAACGGCCT | |
| Halo_BC56 | CCACCGCTGAGCAATAACTA ACCCATTGCGAT | 159 |
| | CGTAGATGAGTCAACGGCCT | |
| Halo_BC57 | CCACCGCTGAGCAATAACTA TCACCACCCTAT | 160 |
| | CGTAGATGAGTCAACGGCCT | |
| Halo_BC58 | CCACCGCTGAGCAATAACTA GGTCTTCACTTC | 161 |
| | CGTAGATGAGTCAACGGCCT | |
| Halo_BC59 | CCACCGCTGAGCAATAACTA GTTAGAGATGGG | 162 |
| | CGTAGATGAGTCAACGGCCT | |
| Halo_BC60 | CCACCGCTGAGCAATAACTA TCTTGCACACTC | 163 |
| | CGTAGATGAGTCAACGGCCT | |
| Halo_BC61 | CCACCGCTGAGCAATAACTA TTTTCTCTGCGG | 164 |
| | CGTAGATGAGTCAACGGCCT | |
| Halo_BC62 | CCACCGCTGAGCAATAACTA TCAGCCGAGTTA | 165 |
| | CGTAGATGAGTCAACGGCCT | |
| Halo_BC63 | CCACCGCTGAGCAATAACTA CTCGTGATCAGA | 166 |
| | CGTAGATGAGTCAACGGCCT | |
| Halo_BC64 | CCACCGCTGAGCAATAACTA CCTTTCTCGGAA | 167 |
| | CGTAGATGAGTCAACGGCCT | |
| Halo_BC65 | CCACCGCTGAGCAATAACTA ACGCTAGAGCTT | 168 |
| | CGTAGATGAGTCAACGGCCT | |
| Halo_BC66 | CCACCGCTGAGCAATAACTA TTCCCCGTTTAG | 169 |
| | CGTAGATGAGTCAACGGCCT | |
| Halo_BC67 | CCACCGCTGAGCAATAACTA AGAATCGCAACC | 170 |
| | CGTAGATGAGTCAACGGCCT | |
| Halo_BC68 | CCACCGCTGAGCAATAACTA GGAAGGAACTGT | 171 |
| | CGTAGATGAGTCAACGGCCT | |
| Halo_BC69 | CCACCGCTGAGCAATAACTA CTTGGCATCTTC | 172 |
| | CGTAGATGAGTCAACGGCCT | |
| Halo_BC70 | CCACCGCTGAGCAATAACTA AGGCCGATTTGT | 173 |
| | CGTAGATGAGTCAACGGCCT | |
| Halo_BC71 | CCACCGCTGAGCAATAACTA AACAAAGGGTCC | 174 |
| | CGTAGATGAGTCAACGGCCT | |
| Halo_BC72 | CCACCGCTGAGCAATAACTA CAATTGGTAGCC | 175 |
| | CGTAGATGAGTCAACGGCCT | |
| Halo_BC73 | CCACCGCTGAGCAATAACTA ACCATCGACTCA | 176 |
| | CGTAGATGAGTCAACGGCCT | |
| Halo_BC74 | CCACCGCTGAGCAATAACTA CGTGAGATGAAC | 177 |
| | CGTAGATGAGTCAACGGCCT | |
| Halo_BC75 | CCACCGCTGAGCAATAACTA CCATGGTCTTGT | 178 |
| | CGTAGATGAGTCAACGGCCT | |
| Halo_BC76 | CCACCGCTGAGCAATAACTA AGATATGAGCGC | 179 |
| | CGTAGATGAGTCAACGGCCT | |
| Halo_BC77 | CCACCGCTGAGCAATAACTA GTGTGACAGAGT | 180 |
| | CGTAGATGAGTCAACGGCCT | |
| Halo_BC78 | CCACCGCTGAGCAATAACTA ATTGTGTGACGG | 181 |
| | CGTAGATGAGTCAACGGCCT | |
| Halo_BC79 | CCACCGCTGAGCAATAACTA CGGTAGTTTGCT | 182 |
| | CGTAGATGAGTCAACGGCCT | |
| Halo_BC80 | CCACCGCTGAGCAATAACTA GGACATGTCCAT | 183 |
| | CGTAGATGAGTCAACGGCCT | |
| Halo_BC81 | CCACCGCTGAGCAATAACTA TTGAGGGAGACA | 184 |
| | CGTAGATGAGTCAACGGCCT | |
| Halo_BC82 | CCACCGCTGAGCAATAACTA CGACATCCTCTA | 185 |
| | CGTAGATGAGTCAACGGCCT | |
| Halo_BC83 | CCACCGCTGAGCAATAACTA TGAGCGAGTTCA | 186 |
| | CGTAGATGAGTCAACGGCCT | |
| Halo_BC84 | CCACCGCTGAGCAATAACTA GACCTTCGGATT | 187 |
| | CGTAGATGAGTCAACGGCCT | |
| Halo_BC85 | CCACCGCTGAGCAATAACTA TGTAGATCCGCA | 188 |
| | CGTAGATGAGTCAACGGCCT | |
| Halo_BC86 | CCACCGCTGAGCAATAACTA TGGCACTCTAGA | 189 |
| | CGTAGATGAGTCAACGGCCT | |
| Halo_BC87 | CCACCGCTGAGCAATAACTA AACAGTAGTCGG | 190 |
| | CGTAGATGAGTCAACGGCCT | |
| Halo_BC88 | CCACCGCTGAGCAATAACTA TCATGCGGAAAG | 191 |
| | CGTAGATGAGTCAACGGCCT | |
| Halo_BC89 | CCACCGCTGAGCAATAACTA TCGAATCGTGTC | 192 |
| | CGTAGATGAGTCAACGGCCT | |
| Halo_BC90 | CCACCGCTGAGCAATAACTA GGTGTATAGCCA | 193 |
| | CGTAGATGAGTCAACGGCCT | |
| Halo_BC91 | CCACCGCTGAGCAATAACTA TTGCAGTGCAAG | 194 |
| | CGTAGATGAGTCAACGGCCT | |
| Halo_BC92 | CCACCGCTGAGCAATAACTA CGATTGCAGAAG | 195 |
| | CGTAGATGAGTCAACGGCCT | |
| Halo_BC93 | CCACCGCTGAGCAATAACTA CCAGACGTTGTT | 196 |
| | CGTAGATGAGTCAACGGCCT | |
| Halo_BC94 | CCACCGCTGAGCAATAACTA TGGTGGCCATAA | 197 |
| | CGTAGATGAGTCAACGGCCT | |
| Halo_BC95 | CCACCGCTGAGCAATAACTA CAGAGTCAATGG | 198 |
| | CGTAGATGAGTCAACGGCCT | |
| Halo_BC96 | CCACCGCTGAGCAATAACTA CCTATCATTCCC | 199 |
| | CGTAGATGAGTCAACGGCCT | |
| Halo_BC97 | CCACCGCTGAGCAATAACTA GAGGTATGACTC | 200 |
| | CGTAGATGAGTCAACGGCCT | |
| Halo_BC98 | CCACCGCTGAGCAATAACTA CTAGGTCAAGTC | 201 |
| | CGTAGATGAGTCAACGGCCT | |
| Halo_BC99 | CCACCGCTGAGCAATAACTA ACTCGGCTTTCA | 202 |
| | CGTAGATGAGTCAACGGCCT | |
| Halo_BC100 | CCACCGCTGAGCAATAACTA TTCACAAGCGGA | 203 |
| | CGTAGATGAGTCAACGGCCT | |

### Methods

In another aspect, provided herein are methods for multiplexed detection and measurement of multiple targets in a sample using affinity reagents that comprise a unique DNA barcode. In some cases, the method comprises contacting affinity reagents comprising unique DNA barcodes to a sample under conditions that promote binding of the affinity reagents to target antigens when present in said sample. The methods provided herein can employ a variety of affinity reagents, including those favored by a user, in a multiplexed set to measure the abundance of their respective targets in a sample. The methods provided herein permit measurement of the levels of proteins or any detectable antigens in high throughput. This method uses available antibodies which enables the user to use those antibodies that have the best specification for purpose. This does not require the user to remain within a closed system such as a proprietary set of aptamers or a set of reagents for which binding data are not public. The method will have a wide dynamic range and can be multiplexed in the thousands.

In cases in which the affinity reagents are antibodies and the targets are antigens, antibodies that are bound to their target antigens can be separated from unbound antibodies. Any method of uniquely detecting and measuring the DNA barcodes can be used. In some embodiments, the DNA barcode associated with the affinity reagent is amplified, such as by polymerase chain reaction (PCR) or another amplification technique, and the amplified barcode DNA is subjected to DNA sequencing to provide a measure of target protein in the contacted sample. In other cases, the DNA barcode is detected using, for example, a nucleic acid array or aptamers.

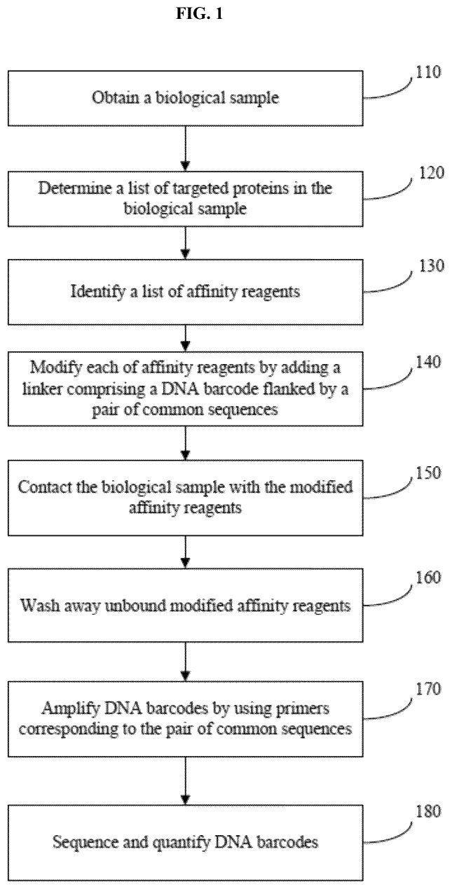

Referring to the flow chart of FIG. 1, the methods in some cases comprise obtaining a biological sample (see step 110). In step 120, the user may define a list of target proteins (or other targets) to be detected and quantified in the sample. In step 130, affinity reagents that specifically recognize each of the targets on the list are prepared by linking a unique barcode to antibodies or aptamers having affinity for those targets.

In some embodiments, protein measurement comprises separating bound antibodies from unbound antibodies. In some cases, the sample is brought into contact with the antibody mix under conditions that promote binding of affinity reagents to their targets if presented in the sample. Unbound antibodies are washed away in step 160.

Any appropriate method can be used to detect and measure binding of affinity reagents to their targets in the sample. For example, referring to step 170 of FIG. 1, PCR-based amplification can be performed directly on the sample using primers that correspond to the sequences that flank the bar code. As described above, the flanking amplifying sequences can comprise nucleotide sequences of CCACCGCTGAGCAATAACTA (SEQ ID NO:2) and CGTAGATGAGTCAACGGCCT (SEQ ID NO:3). By sequencing the resulting amplified DNA, the number of each type of target can be assessed based on the barcode. In other embodiments, linkers containing the barcodes are released from the samples by photo cleavage or a chemical cleavage, and then collected and used to run a PCR reaction as above. The resulting amplified DNA is subjected to DNA sequencing to assess the number of each type of target based on the barcode. In yet other embodiments, the linkers containing both the barcodes and fluorescent tag are released from the samples by photo cleavage, and then collected. They are then used to hybridize to a DNA microarray that specifically recognizes the barcodes.

The terms “quantity”, “amount” and “level” are synonymous and generally well-understood in the art. The terms as used herein may particularly refer to an absolute quantification of a target molecule in a sample, or to a relative quantification of a target molecule in a sample, i.e., relative to another value such as relative to a reference value or to a range of values indicating a base-line expression of the biomarker. These values or ranges can be obtained from a single subject (e.g., human patient) or aggregated from a group of subjects. In some cases, target measurements are compared to a standard or set of standards.

In a further aspect, provided herein are methods for detecting and quantifying a subject's immune response to a disease (e.g., cancer, autoimmune disorder) or infectious agent such as a pathogenic microorganism. In such cases, affinity reagents are selected for their affinity for molecular targets associated with a particular disease or infectious agent. Advantageously, the affinity reagents described herein are well suited for multiplexed screening of a sample for many different infections. For example, one may assay a sample for many infections simultaneously to see which induced an immune response and to which infection-associated proteins triggered the response. Samples appropriate for use according to the methods provided herein include biological samples such as, for example, blood, plasma, serum, urine, saliva, tissues, cells, organs, organisms or portions thereof (e.g., mosquitoes, bacteria, plants or plant material), patient samples (e.g., feces or body fluids, such as urine, blood, serum, plasma, or cerebrospinal fluid), food samples, drinking water, and agricultural products.

In certain embodiments, affinity reagents described herein are used to detect and, in some cases, monitor a subject's immune response to an infectious pathogen. By way of example, pathogens may comprise viruses including, without limitation, flaviruses, human immunodeficiency virus (HIV), Ebola virus, single stranded RNA viruses, single stranded DNA viruses, double-stranded RNA viruses, double-stranded DNA viruses. Other pathogens include but are not limited to parasites (e.g., malaria parasites and other protozoan and metazoan pathogens (plasmodia species, leishmania species, schistosoma species, trypanosoma species)), bacteria (e.g., Mycobacteria, in particular, M. tuberculosis, Salmonella, Streptococci, E. coli, Staphylococci), fungi (e.g., candida species, aspergillus species, Pneumocystis jiroveciiand other Pneumocystis species), and prions. In some cases, the pathogenic microorganism, e.g. pathogenic bacteria, may be one which causes cancer in certain human cell types.

In certain embodiments, the methods detect viruses including, without limitation, the human-pathogenic viruses such Zika virus (e.g., Zika strain from the Americas, ZIKV), yellow fever virus, and dengue virus serotypes 1 (DENV1) and 3 (DENV3), and closely related viruses such as the chikungunya virus (CHIKV).

The terms “detect” or “detection” as used herein indicate the determination of the existence, presence or fact of a target molecule in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

### Articles of Manufacture

In another aspect, provided herein are articles of manufacture useful for detecting target molecules, including infection-associated or disease-associated molecules (e.g., cancer associated). In certain embodiments, the article of manufacture is a kit for detecting an immune response to a pathogen, where the kit comprises a plurality of affinity reagents, each of which comprises a linked DNA barcode, and one or more of reagents to amplify DNA barcodes using polymerase chain reaction. Preferably, the linked DNA barcode is flanked by a pair of amplifying nucleotide sequences, and each affinity reagent has a different identifying barcode sequence from other affinity reagents. Optionally, a kit can further include instructions for performing the detection and/or amplification methods described herein.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The indefinite articles “a” and “an,” as used herein in the specification and in the claims, unless clearly indicated to the contrary, should be understood to mean “at least one.”

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Unless otherwise indicated, any nucleic acid sequences are written left to right in 5′ to 3′ orientation; amino acid sequences are written left to right in amino to carboxy orientation, respectively.

Schematic flow charts included are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLE

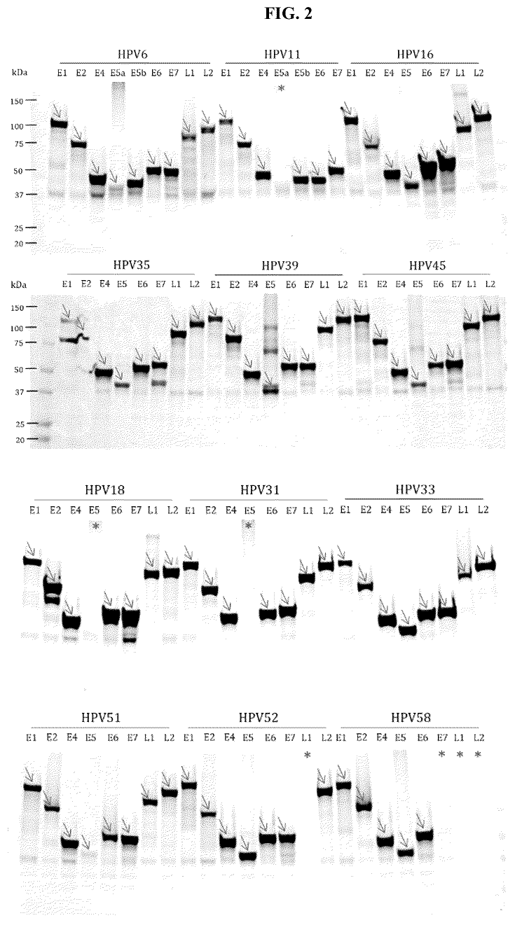

To develop a quantitative, multiplexed, bar-coded antigen library for detection of immune responses in pathogen induced cancers, we cloned 97 HPV genes from the HPV strains 6, 16, 18, 31, 33, 35, 39, 45, 51, 52, and 58 into the pJFT7-3XFLAG-Halo vector. This vector includes two fusion tags 3XFLAG and Halo fusion. As shown in FIG. 2, the HPV proteome was expressed in a cell-free human IVT system with a 97% success rate. Except for the HPV11 E5a and HPV 31 E5 antigens, full-length proteins for the HPV proteome were successfully expressed.

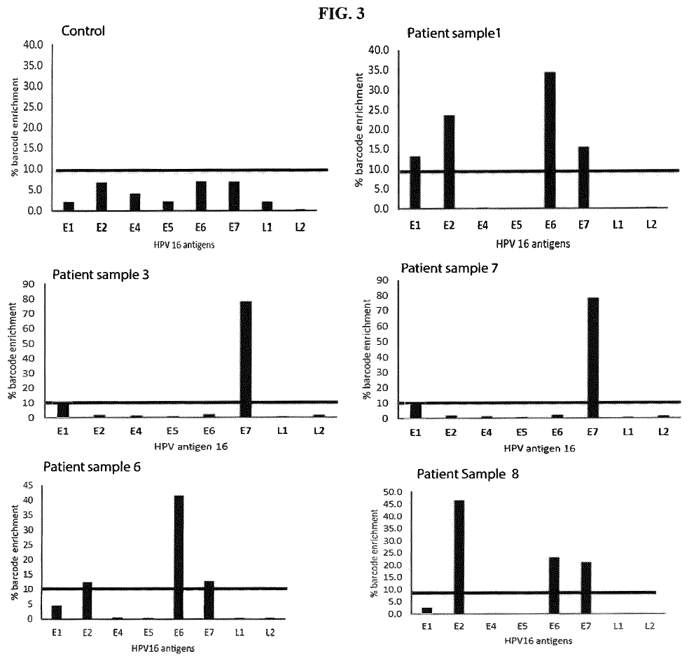

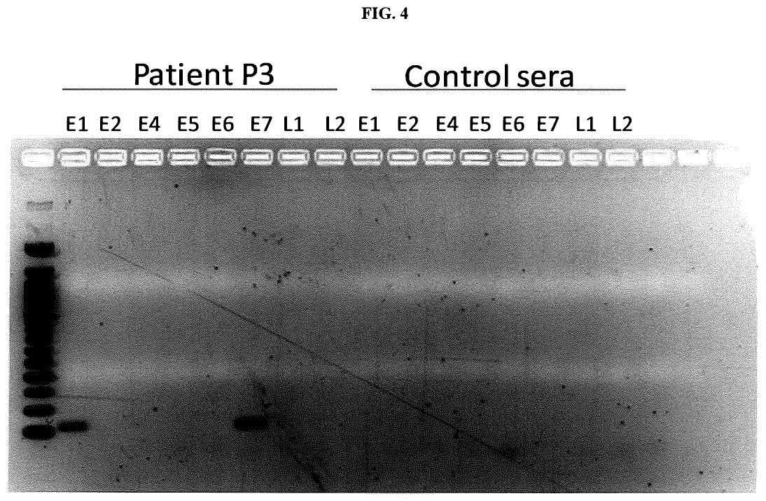

Unique DNA barcodes (attached to Halo ligand) were appended to 20 antigens from HPV strains 16, 18 (high risk HPV strains) and 6 (a low risk HPV strain). After capturing the expressed and barcoded HPV antigens with FLAG magnetic beads we combined all the HPV antigens into a single protein cocktail. This barcoded protein cocktail was then probed against 10 HPV infected OPC patient samples and 10 control samples. After capturing in protein, A/G magnetic beads we amplified the barcodes and ran the samples on NextSeq after multiplexing. From our sequencing run we obtained 450K reads per sample with 71% mapping ratio to our barcodes. The normalized percentage of each barcode showed distinct enrichment of certain HPV antigens in the OPC patient samples (FIG. 3). In contrast, most of the control samples showed only less than 10% barcode enrichment for the HPV antigens. This clearly demonstrates that the barcoded HPV proteome can be utilized to quantify the immune responses for certain HPV antigens in OPC patient sera. We observed a heterogeneous immune response for the HPV positive OPC serum sample, where antibodies were detected for E1, E2, E6 and E7 HPV 16 antigens. We also detected similar patterns for antibody profiles when we amplified our unique barcodes with barcode specific PCR primers (FIG. 4).

We claim:

1. A composition comprising a plurality of modified affinity reagents, wherein each affinity reagent of the plurality comprises a linker, the linker comprising a unique identifying nucleotide sequence relative to other affinity reagents of the plurality flanked by a first amplifying nucleotide sequence and a second amplifying nucleotide sequence, wherein:
(a) the first and second amplifying nucleotide sequence are independently selected from SEQ ID NOs: 2 or 3; or
(b) the linker comprises one of SEQ ID NOs. 105-203.

2. The composition of claim 1, wherein affinity reagents of the plurality are antibodies, peptide aptamers, or nucleic acid aptamers.

3. The composition of claim 1, wherein the linker further comprises a cleavable protein photocrosslinker or a fluorescent moiety.

4. The composition of claim 1, wherein the wherein the identifying nucleotide sequence has a length of about 10 nucleotides to about 20 nucleotides.

5. The composition of claim 1, the wherein the identifying nucleotide sequence has a length of about 12 nucleotides.

6. The composition of claim 1, wherein the identifying nucleotide sequence comprises about 50% of AT base pairs and about 50% of GC base pairs.

7. A method for high throughput target molecule identification and quantification, comprising:
contacting a sample with a plurality of modified affinity reagents under conditions that promote binding of modified affinity reagents of the plurality to target molecules if present in the contacted sample, wherein each modified affinity reagent of the plurality is coupled to a linker comprising an identifying nucleotide sequence flanked on each end by an amplifying nucleotide sequence, wherein (a) the amplifying nucleotide sequence comprises SEQ ID NO: 2 or 3 or (b) the linker comprises one of SEQ ID NOs: 105-203;
removing unbound modified affinity reagent from the contacted sample; and
amplifying and sequencing the identifying nucleotide sequence coupled to each bound modified affinity reagent whereby the target molecules are identified and quantified based on detection of identifying nucleotide sequences of the bound modified affinity reagents.

8. The method of claim 7, wherein the affinity reagent is an antibody or an aptamer.

9. The method of claim 8, wherein the affinity reagent is an antibody and wherein the linker is coupled to a region of the antibody that is not an antigen binding region.

10. The method of claim 8, wherein the affinity reagent is an antibody and wherein the linker is coupled to a fragment crystallizable region (Fc region) of the antibody.

11. The method of claim 7, wherein the identifying nucleotide sequence has a length of about 10 nucleotides to about 20 nucleotides.

12. The method of claim 7, wherein the identifying nucleotide sequence has a length of about 12 nucleotides.

13. The method of claim 7, wherein the identifying nucleotide sequence comprises about 50% of AT base pairs and about 50% of GC base pairs.

14. The method of claim 7, wherein the amplifying sequence has a length ranging from 20 to 30 base pairs.

15. The method of claim 7, wherein the linker further comprises a fluorescent protein or a cleavable protein photocrosslinker.

16. A kit for high throughput protein quantification, comprising the composition comprising a plurality of modified affinity reagents of claim 1, the plurality comprising X modified affinity reagent(s), wherein:
X is equal to or greater than 1.
